(19) United States
(12) Patent Application Publication
(10) Pub. No.: US 2023/0026212 A1
(43) Pub. Date: Jan. 26, 2023

(54) ELECTROCARDIOGRAM ELECTRODE APPLICATION SYSTEM

(71) Applicant: Spaulding Medical, LLC, West Bend, WI (US)

(72) Inventors: Don Brodnick, Cederberg, WI (US); Randol R. Spaulding, West Bend, WI (US); Jay Mason, Reno, NV (US); Mark Geisler, Franklin, WI (US)

(21) Appl. No.: 17/850,500

(22) Filed: Jun. 27, 2022

Related U.S. Application Data
(60) Provisional application No. 63/215,317, filed on Jun. 25, 2021.

(51) Int. Cl. A61B 5/28; A61B 5/00; A61B 5/318

(52) U.S. Cl. CPC A61B 5/28; A61B 5/0006; A61B 5/318; A61B 2562/08; A61B 2562/222

(57) ABSTRACT

An electrocardiogram electrode application system includes electrodes that detect electrical signals connected by a single connecting wire and transmitters capable of wirelessly transmitting the detected electrical signals to a computing device for analysis.

### CROSS-REFERENCE TO RELATED APPLICATION(S)

This application claims priority to U.S. Application No. 63/215,317, filed on Jun. 25, 2021, the disclosure of which is hereby incorporated by reference in its entirety.

### BACKGROUND

An electrocardiogram system records electrical signals from a patient's heart to check for different heart conditions. Electrodes are placed on the patient to detect the electrical signals. Wires are connected to each electrode to transmit the signals to a computing device for analysis.

A conventional electrocardiogram system includes ten electrodes placed on the patient's limbs and chest. An individual wire connects to each electrode to send the detected signals to a computing device. The heart's electric potential is measured from the electrodes and recorded over a period of time.

### SUMMARY

In general terms, this disclosure is directed to an electrocardiogram electrode application system. In a non-limiting example, the electrocardiogram electrode application system includes electrodes that detect electrical signals connected by a single connecting wire and transmitters capable of wirelessly transmitting the detected electrical signals to a computing device for analysis.

One aspect is a method comprising detecting electrical signals of a patient by two or more electrodes connected by a single connecting wire and placed on the patient; receiving the electrical signals by two or more transmitters connected to the two or more electrodes; and wirelessly transmitting the electrical signals by the two or more transmitters.

In one example, the two or more transmitters are universal transmitters capable of connecting to any of the two or more electrodes. In another example, wirelessly transmitting the electrical signals by the two or more transmitters comprises transmitting the electrical signals to an electrocardiogram monitoring device. In yet another example, the method further comprises determining, by the two or more transmitters, the identity of the two or more electrodes. In an additional example, determining the identity of the two or more electrodes comprises observing a voltage on a 4 bit coded identity of the electrodes. In a further example, the method further comprises transmitting the identity of the two or more electrodes.

Another aspect is a system comprising two or more electrodes connected by a single connecting wire and placed on a patient, wherein the two or more electrodes are operable to detect electrical signals of the patient; and two or more transmitters connected to the two or more electrodes, wherein the two or more electrodes are operable to receive the electrical signals, and wirelessly transmit the electrical signals.

In one example, the two or more transmitters are universal transmitters capable of connecting to any of the two or more electrodes. In another example, to wirelessly transmit the electrical signals by the two or more transmitters comprises to transmit the electrical signals to an electrocardiogram monitoring device. In yet another example, the two or more electrodes are further operative to determine the identity of the two or more electrodes. In an additional example, to determine the identity of the two or more electrodes comprises to observe a voltage on a 4 bit coded identity of the electrodes. In a further example, the two or more electrodes transmit the identity of the two or more electrodes. In another example, the two or more electrodes comprise a substrate and a conductive ink printed on the substrate, the conductive ink comprising: a sensor conductor to detect electrical signals of the patient the electrode is connected to, a sensor conductor lead, and a connecting wire lead. In an additional example, the system further comprises a reference electrode operable to provide a reference voltage to the two or more electrodes.

A further aspect is a system comprising: a memory storage; and a processing unit coupled to the memory storage, wherein the processing unit is operative to: detect electrical signals of a patient by two or more electrodes connected by a single connecting wire and placed on the patient; receive the electrical signals by two or more transmitters connected to the two or more electrodes; and wirelessly transmit the electrical signals by the two or more transmitters.

In one example, the two or more transmitters are universal transmitters capable of connecting to any of the two or more electrodes. In another example, wirelessly transmitting the electrical signals by the two or more transmitters comprises transmitting the electrical signals to an electrocardiogram monitoring device. In yet another example, the processing unit is further operable to determine the identity of the two or more electrodes. In an additional example, to determine the identity of the two or more electrodes comprises observing a voltage on a 4 bit coded identity of the electrodes. In a further example, the processing unit is further operable to transmit the identity of the two or more electrodes.

### BRIEF DESCRIPTION OF THE DRAWINGS

FIG. 1 illustrates an electrocardiogram electrode application system according to an example;

FIG. 2 illustrates a block diagram of a transmitter according to an example;

FIG. 3 illustrates a flow chart of a method for operating an electrocardiogram electrode application system according to an example;

FIG. 4 illustrates a side view of an example transmitter of an electrocardiogram electrode application system according to an example;

FIG. 5 illustrates a top view of an example transmitter of an electrocardiogram electrode application system according to an example;

FIG. 6 illustrates an exploded top view of a transmitter and an electrode of an electrocardiogram electrode application system according to an example;

FIG. 7 illustrates an electrode of an electrocardiogram electrode application system according to an example;

FIG. 8 illustrates a reference electrode of an electrocardiogram electrode application system according to an example;

FIG. 9 illustrates a side view of a transmitter in an open position and an example electrode disconnected from the transmitter according to an example;

FIG. 10 illustrates a side view of a transmitter in a closed position and an electrode connected to the transmitter according to an example;

FIG. 11 illustrates electrodes of an electrocardiogram electrode application system according to an example; and

FIG. 12 illustrates a configuration of electrodes of an electrocardiogram electrode application system along a single connection wire according to an example.

### DETAILED DESCRIPTION

Various examples will be described in detail with reference to the drawings, wherein like reference numerals represent like parts and assemblies throughout the several views. Reference to various example does not limit the scope of the claims attached hereto. Additionally, any examples set forth in this specification are not intended to be limiting and merely set forth some of the many possible examples for the appended claims.

The present disclosure describes an example electrocardiogram electrode application system including electrodes that detect electrical signals connected by a single connecting wire and transmitters that connect to the electrodes. The transmitters are capable of wirelessly transmitting the detected electrical signals to a computing device for analysis. The transmitters can connect to any electrode placed on a patient and transmit the electrical signals detected by the electrode the transmitter is connected to. The transmitters can identify the electrode the transmitter is connected to including identifying the location of the electrode on the patient. The transmitter can include the location of the electrode in its transmission of electrical signals to the computing device.

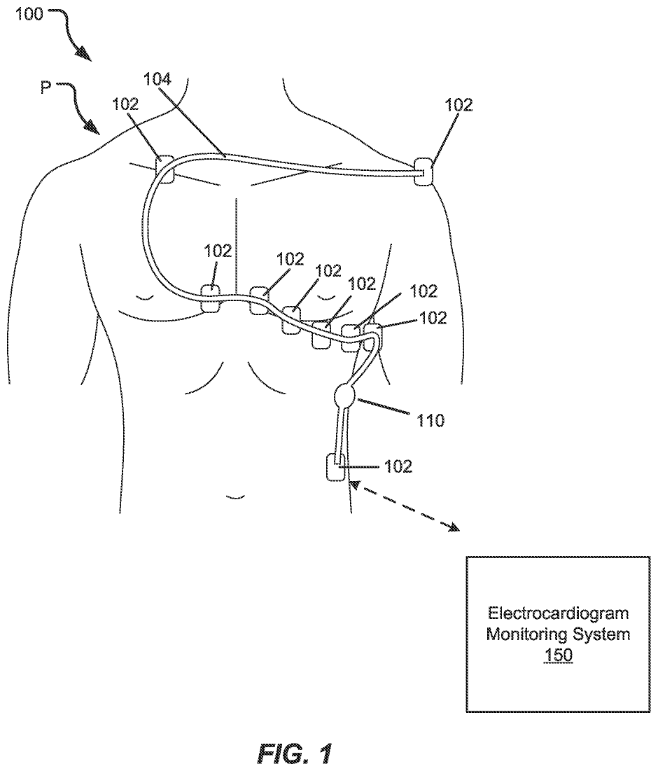

FIG. 1 illustrates an electrocardiogram (ECG) electrode application system 100 according to an example. The example ECG electrode application system 100 may include electrodes 102 connected by a single connecting wire 104, and a reference electrode 110 additionally connected by the connecting wire 104. The ECG electrode application system 100 may be attached to a patient P. The electrodes 102 in the ECG electrode application system 100 may detect electrical changes of the heart of patient P. The ECG electrode application system 100 may also include transmitters that may amplify, process, and/or transmit the electrical signals the electrodes 102 detect. The transmitters may be able to connect to any of the electrodes 102 and operate correctly. The transmitters may transmit the electrical signals to an ECG monitoring system 150. The ECG monitoring system 150 may process, display, and/or store the received signals. The ECG monitoring system 150 may be a personal computer, a mobile device, a medical device, or the like.

The configuration of the ECG electrode application system 100 on the patient P is an example configuration, and the ECG electrode application system 100 may be configured differently on the patient P in other examples because the electrodes 102 may be arranged in any position that allows the electrodes to detect the electrical signals of the patient P's heart. For example, the electrodes 102 may be positioned on the patient P's limbs, back, head, neck, and the like. Additionally, the ECG electrode application system 100 may contain more or fewer electrodes 102. For example, three electrodes may be used for a 3-lead ECG, five electrodes may be used for a 5-lead ECG, ten electrodes may be used for a 12-lead ECG, and the like.

The reference electrode 110 may reduce interference so the electrodes 102 may accurately detect the electrical changes of the patient P. In an example, connecting wire 104 and reference electrode 110 provide a reference voltage for the ECG electrode application system 100. In some examples, the reference electrode 110 may not be included in the ECG electrode application system 100. In these examples, the connecting wire 104 may provide the reference voltage. An electrode may be selected as a chassis ground, and all other electrodes may measure detected electrical signals against the chosen electrode.

In an example, the electrodes 102 and the connecting wire 104 are a single piece distributed on a deployment card release liner. A user may connect transmitters to the electrodes 102, remove the electrodes from the deployment card release liner, and place the electrodes 102 on a patient, such as patient P. The deployment card release liner may be a sheet that the electrodes 102 contact, and the electrodes 102 may be positioned in an order for positioning on the patient P. In the illustrated example, the reference electrode 110 is positioned between two electrodes on the left side of patient P's abdomen. In other examples, the reference electrode 110 may be located in a different position on patient P's body. The reference electrode 110 can be located in any position that allows the single connecting wire 104 to connect to the other electrodes 102.

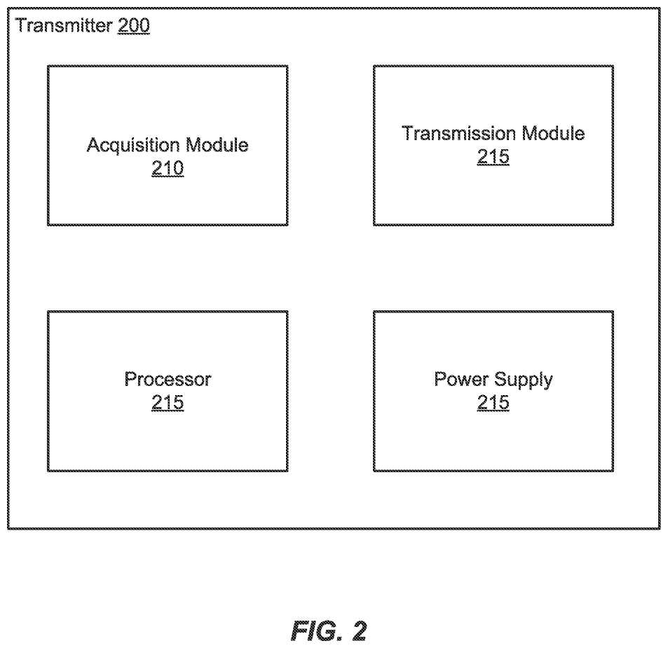

FIG. 2 illustrates a block diagram of a transmitter 200 that may connect to an electrode 102 of the ECG electrode application system 100, shown in FIG. 1. The transmitter 200 may be a universal transmitter that can interchangeably connect to any of the electrodes 102. The transmitter may include an acquisition module 210, a transmission module 215, a processor 220, and a power supply 225.

The acquisition module 210 may receive the electrical signals detected by the connected electrode. The transmission module 215 may transmit the data, such as the received electrical signals, to external devices, such as the ECG monitoring system 150. The transmission module 215 may transmit the data via radio frequency (RF) components, optical components, audio components, and the like. For example, the transmission module may include an RF component for the transmission module 215 to transmit the electrical signals via radio waves such as Bluetooth®, medical band Bluetooth®, and the like. The transmission module 215 may include components to transmit the data wirelessly and/or via a wired connection. The transmission module 215 may transmit the data in real time or substantially real time and/or cause the transmitter 200 to store the data and transmit the data later. For example, the transmitters 200 may be removed from a patient before the transmission module 215 transmits the data.

The processor 220 may cause the acquisition module 210 and the transmission module 215 to operate. For example, the processor 220 may cause the acquisition module 210 to receive data (e.g., electrical signals from the electrodes 102), cause the data to be sent to the transmission module 215, and cause the transmission module 215 to transmit the data. The processor 220 may also process the received data and cause the transmission module 215 to transmit the processed data. For example, the ECG monitoring system 150 may require the data to be formatted before the receiving the data, so the processor 220 may format the data before the transmission module 215 transmits the data to the ECG monitoring system 150.

The power supply 225 may power the transmitter 200, including the acquisition module 210, the transmission module 215, and the processor 220. The power supply 225 may be a battery, a capacitor, or the like. The power supply 225 may be rechargeable. For example, the power supply 225 may be wirelessly rechargeable, and the power supply 225 can be charged before the transmitter 200 is connected to a patient, while the transmitter 200 is connected to the patient, and/or after the transmitter 200 is connected to a patient.

Multiple transmitters 200 may be used for a patient, such as patient P. For example, a transmitter 200 may be connected to each electrode 102 attached to the patient P. Therefore, each transmitter 200 may receive electrical signals and transmit the signals to a computing device, such as the ECG monitoring system 150. Each transmitter 200 may identify which electrode the transmitter 200 is connected to, which will be described in more detail herein with respect to FIG. 11. The identity of the electrodes may include the position that the electrode is placed on a patient and the location of electrode with respect to other electrodes in the electrocardiogram electrode application system. Based on the identity of the electrode each transmitter 200 is connected to, one electrode 102 may perform long distance transmission. For example, if the transmitter 200 is attached to a specific electrode, such as a left leg (LL) electrode or left arm (LA) electrode, the transmitter 200 may perform long distance transmission. The transmission module 215 of the transmitter 200 performing long distance transmission may transmit data to a computing device such as the ECG monitoring system 150. The other transmitters 200 may transmit data to the transmitter 200 performing long distance transmission, so the transmitter 200 performing long distance transmission will forward the data to the ECG monitoring system 150. Thus, a single transmitter may transmit the data to external devices, and the other transmitters may transmit data to the single transmitter.

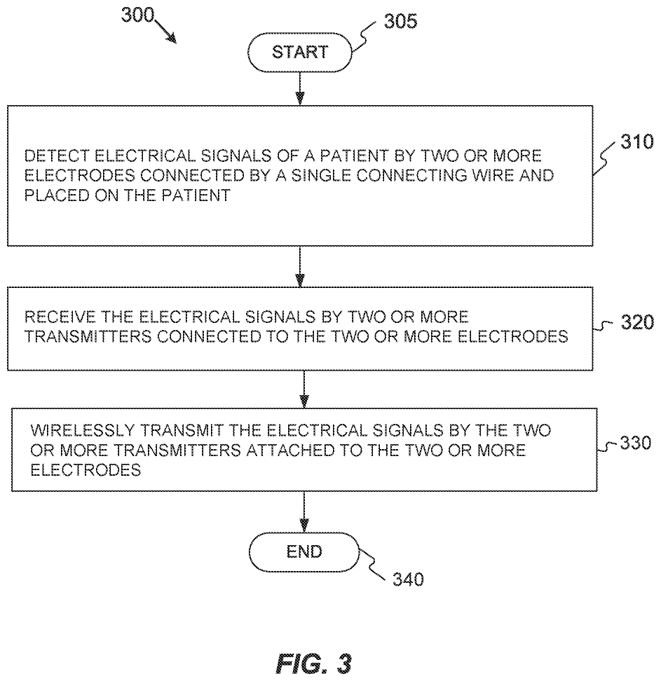

FIG. 3 illustrates a flow chart of a method 300 for operating an ECG electrode application system, such as ECG electrode application system 100 shown in FIG. 1, according to an example. The method 300 may begin at starting block 305 and proceed to operation 310, In operation 310, electrical signals of a patient may be received from two or more electrodes connected by a single connecting wire and placed on the patient. For example, the electrodes 102 receive electrical signals of the patient P.

In operation 320, two or more transmitters connected to the two or more electrodes may receive the electrical signals. For example, the electrodes 102 are connected to transmitters 200, and the transmitters 200 receive the electrical signals.

In operation 330, the transmitters may wirelessly transmit the electrical signals. For example, the transmitters 200 transmit the electric signals to the ECG monitoring system 150. The method 300 concludes at ending block 340.

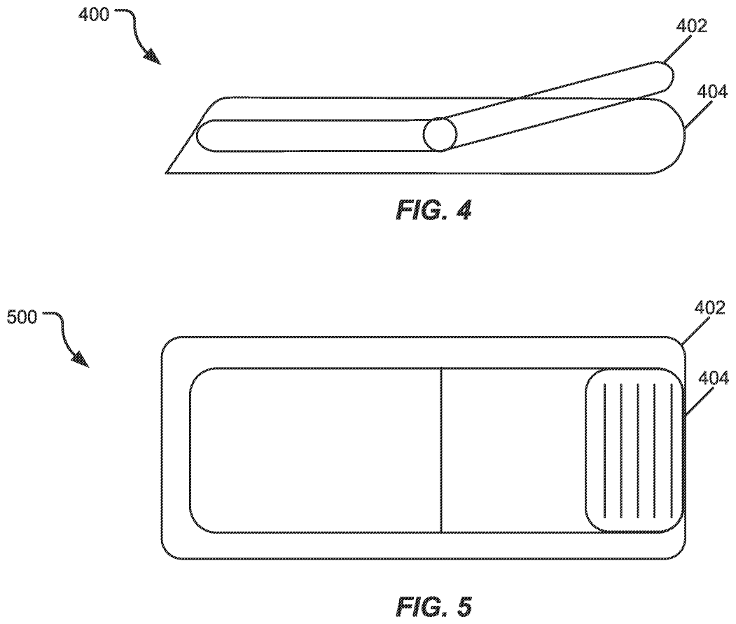

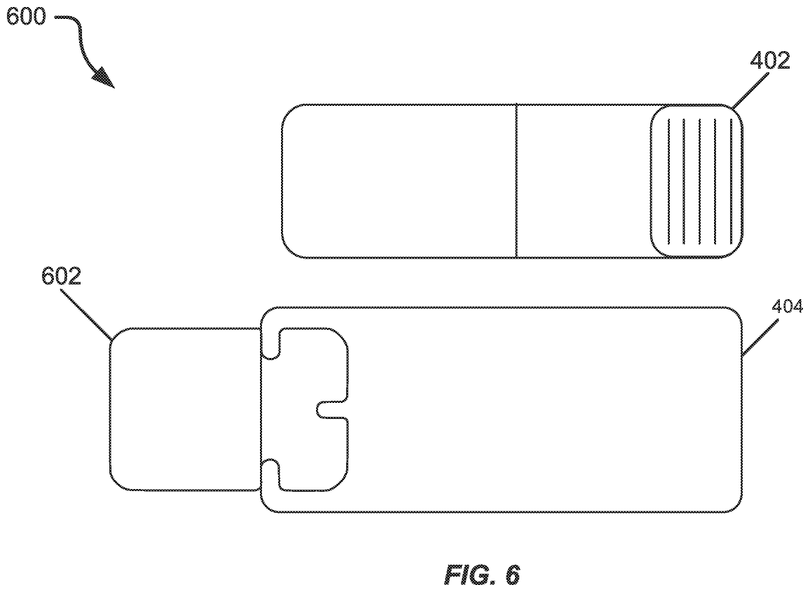

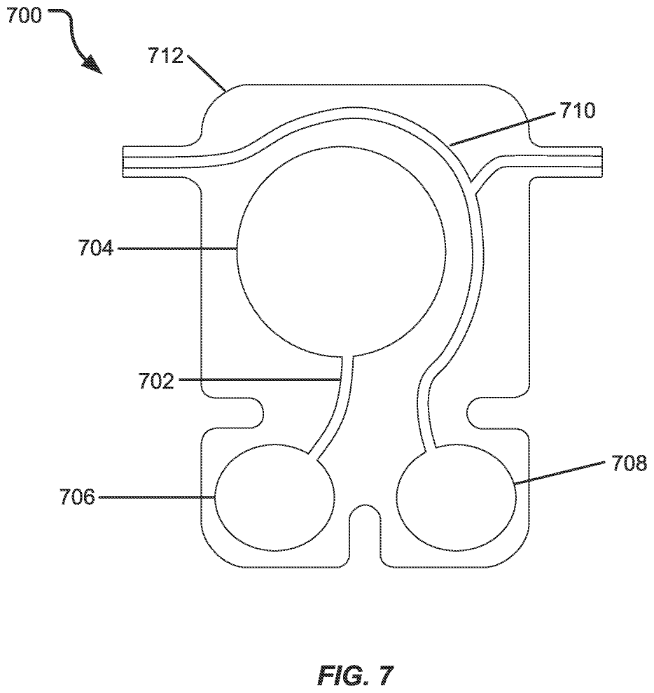

FIG. 4 illustrates a side view of a transmitter 400 of an ECG electrode application system, such as the ECG electrode application system 100 shown in FIG. 1. FIG. 5 illustrates a top view 500 of the transmitter 400. FIG. 6 illustrates an exploded top view 600 of the transmitter 400.

The transmitter 400 may connect to an electrode , such as electrode 102 shown in FIG. 1, and may amplify, process, and/or transmit the electrical signals detected by the electrode the transmitter is connected to. The transmitter 400 may contain a circuit board, a battery, and/or other electronics. In an example, the transmitter 400 may send the electrical signal via Bluetooth® transmission or another wireless transmission method. In the illustrated example, the transmitter 400 may have a clamp 402 in a closed position and a body 404. The clamp 402 may be spring loaded to cause the clamp 402 to remain in the closed position unless the clamp 402 is depressed. For example, the clamp 402 may be depressed by thumb pressure to be in an open position to connect to an electrode. The transmitter 400 can be connected to an electrode while the electrode is attached to a deployment card release liner. A transmitter 400 with a spring loaded clamp 402 connected to an electrode may stay connected until the clamp 402 is depressed to remove the transmitter 400 from the electrode. In an example, the connected transmitter and electrode can be positioned on a patient such as patient P shown in FIG. 1. In an example, the transmitter 400 may be charged wirelessly. The transmitter 400 may also be referred to herein as an ECG module.

FIG. 6 also illustrates a substrate 602. The substrate 602 may be part of an electrode such as the electrodes 102 shown in FIG. 1. In the illustration, the spring clamp 402 of the transmitter 400 is removed from the body of the transmitter to illustrate the connection of the transmitter 400 and the substrate 602. In examples, the clamp 402 is not removed from the transmitter 400 and is depressed to the open position when the transmitter 400 is connected and removed from the substrate 602.

In an example, the clamp 402 closes over the substrate 602 to keep the transmitter 400 and substrate 602 connected. The substrate 602 may have three notches that will align with three ridges in the transmitter to ensure a reliable and/or secure connection, both mechanically and electrically.

FIG. 7 illustrates an example electrode 700 of an ECG electrode application system 100 according to an example. The electrode 700 may be any one of the electrodes 102 shown in FIG. 1. The electrode 700 may include a substrate 712 and conductive ink 702 printed on the substrate 712. In an example, the substrate is 0.5 mm thick. The conductive ink 702 can be printed in any desired pattern to allow the electrode 700 to operate. In the illustrated example, the conductive ink 702 is printed to operate as a basic electrode having a larger sensor conductor 704 connected to a pad 706 for electrical connection to a transmitter such as transmitter 200 shown in FIG. 2 and transmitter 400 shown in FIG. 4, FIG. 5, and FIG. 6. A second pad 708 made of the conductive ink 702 allows for an electrical connection between the electrode 700 and the wire 710 that passes from electrode to electrode such as the connecting wire 104 shown in FIG. 1. The conductive ink 702 may be on a side of the substrate facing the patient, so when the electrode 700 is attached to a patient, the conductive ink 702 contacts the patient. The side of the substrate 712 facing away from the patient may be used for printing information, legends, and markings.

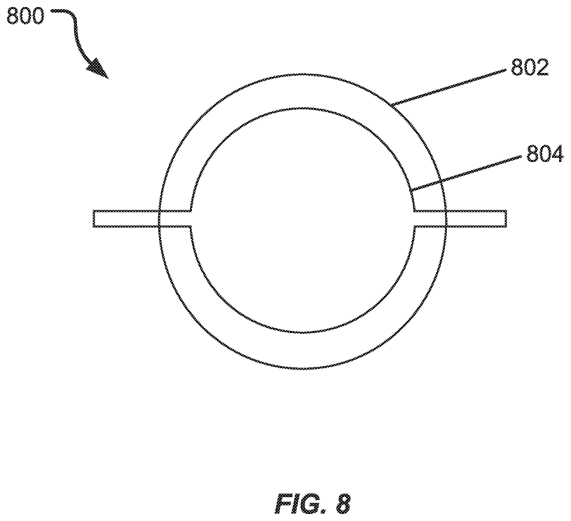

FIG. 8 illustrates an example of a reference electrode 800 of the ECG electrode application system 100 according to an example. The reference electrode 800 may be the reference electrode 110 as shown in FIG. 1. The reference electrode 800 may be placed at any position in the electrocardiogram electrode application system. In examples, the reference electrode 800 needs no electronics and no transmitter is connected to the reference electrode 800. The reference electrode 800 may include a substrate 802 and conductive ink 804.

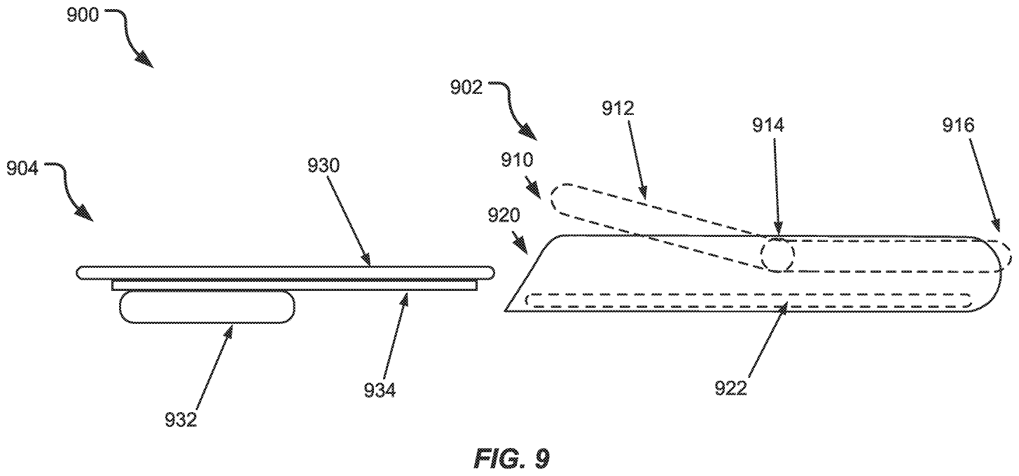

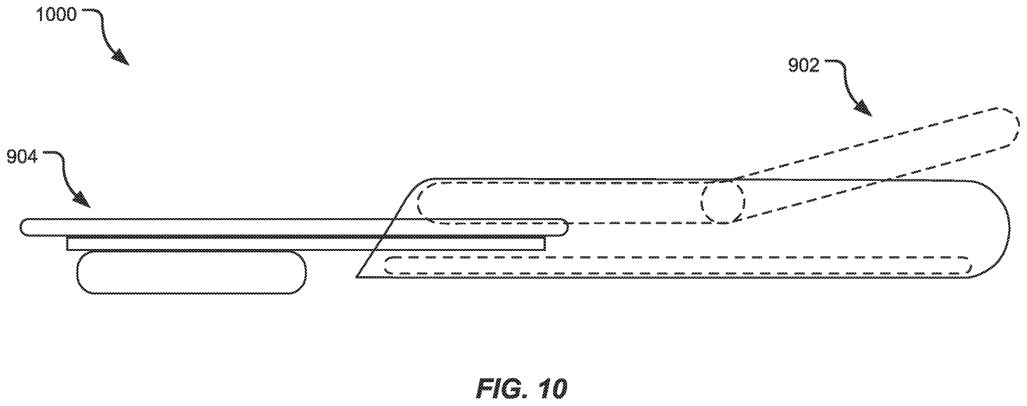

FIG. 9 is a side view illustration 900 of a transmitter 902 and an electrode 904 of an electrocardiogram electrode application system according to an example. The electrode 904 may include a substrate 930, hydrogel 932 for placing electrode 904 on a patient, and conductive ink 934, In an example, the hydrogel 932 is located on the large sensor conductor such as sensor conductor 704 shown in FIG. 7 to allow electrode 904 to detect electrical signals of the patient. In other examples, another substance may be used to connect electrode 904 to a patient. The transmitter 902 may include a clamp 910 and a body 920. The clamp 910 may include a hinge 914 that allows the clamp 910 to move between a closed position and an open position and a thumb grip 916 of the clamp that allows the clamp to be depressed to move a clamping portion 912 from the closed position to the open position. The body 920 nay include a circuit board 922. The circuit board 922 may include the components of the transmitter 200 shown in FIG. 2, including the acquisition module 210, the transmission module 215, the processor 220, and the power supply 225. The circuit board may include any electronics to be able to amplify and/or transmit the electrical signals detected by the electrode 904. In the illustrated example 900, the clamp of transmitter 902 is in the open position to receive the electrode 904. For example, a user may be holding the clamp in the open position by depressing the clamp at the thumb grip.

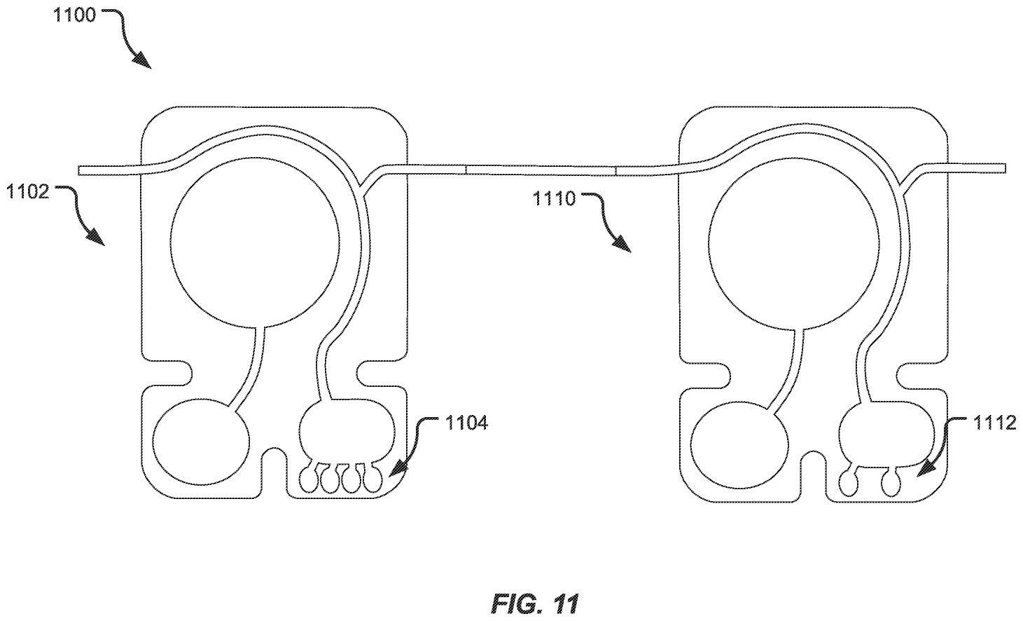

FIG. 10 is an illustration 1000 of the transmitter 902 connected to the electrode 904. For example, as shown in FIG. 9, a user may have depressed the clamp at the thumb grip and connected transmitter 902 to electrode 904. The user may release the thumb grip and the clamp will be in the closed position with the electrode connected as shown in FIG. 10. In the illustrated example 1000, the conductive pads of the electrode, such as pads 706 and 708 shown in FIG. 7, are in contact with the transmitter circuit board.

FIG. 11 is an illustration 1100 of electrodes 1102 and 1110 of an electrocardiogram electrode application system according to an example. Electrodes 1102 and 1110 are examples of different configurations of conductive ink such as the basic configuration of conductive ink 702 shown in FIG. 7. Electrode 1102 illustrates a 4 bit coded identity 1104 printed in conductive ink, and electrode 1110 illustrates a 4 bit coded identity 1112 printed in conductive ink. The 4 bit coded identity 1104 allows a transmitter connected to electrode 1102 to determine the identity of electrode 1102. The identity of electrode 1102 may include the position that the electrode is placed on a patient and the location of electrode 1102 with respect to other electrodes in the electrocardiogram electrode application system. In an example, a transmitter observes the voltage on the 4 bit coded identity to determine the identity of electrode 1102. The 4 bit coded identity 1104 may be used to allow the transmitters of the electrocardiogram electrode application system to be identical and interchangeable and placed at any electrode placed on a patient. The coded identity may include more or fewer bits in other examples.

A transmitter connected to the electrode 1102 may transmit the 4 bit coded identity 1104 and/or the identity of the electrode 1102, such as to the ECG monitoring system 150 shown in FIG. 1. Thus, the ECG monitoring system 150 may use the 4 bit coded identity 1104 and/or the identity of the electrode 1102 for processing, displaying, and storing received data. A transmitter connected to the electrode 1110 may transmit the four bit coded identity 1112 and/or the identity of the electrode 1110, such as to the ECG monitoring system 150. Thus, the ECG monitoring system 150 may use the 4 bit coded identity 1112 and/or the identity of the electrode 1104 for processing, displaying, and storing received data.

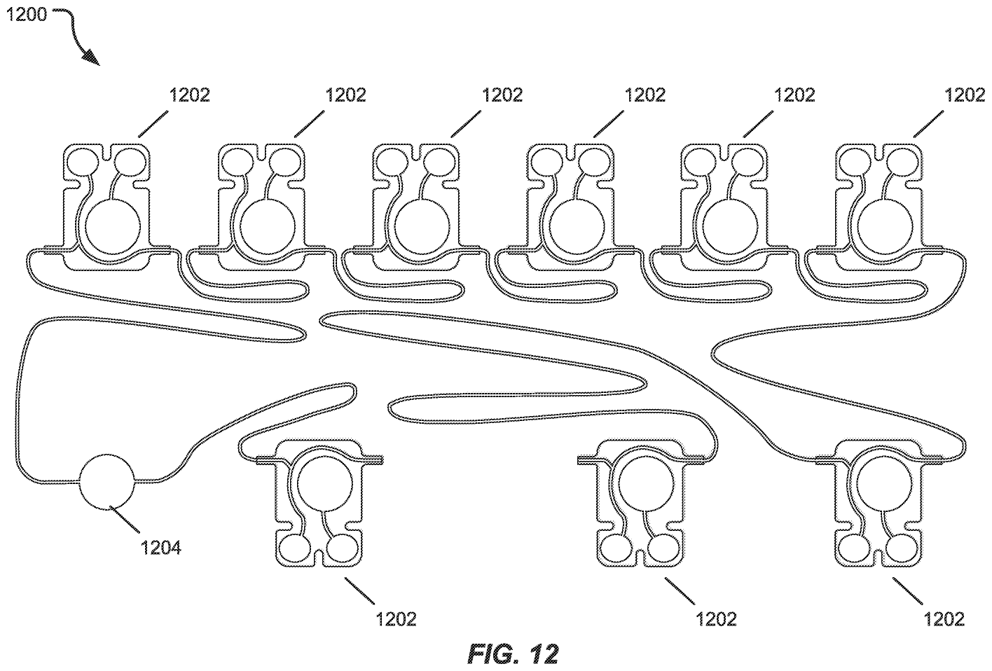

FIG. 12 illustrates an example a configuration of electrodes 1200 of the ECG electrode application system 100 along a connection cable 1206 according to an example. The configuration of electrodes 1200 includes electrodes 1202. The electrodes 1202 may be a V1 electrode, a V2 electrode, a V3 electrode, a V4 electrode, a V5 electrode, a V6 electrode, a left arm (LA) electrode, a left leg (LL) electrode, a right arm (RA) electrode, a right leg (RL) electrode, and the like. The reference electrode 1204 may be the RL electrode in some examples. The electrodes 1202 may be arranged on a deployment card release liner before being placed on a patient. The configuration of electrodes 1200 may be connected via the single connection cable 1206.

What is claimed is:

1. A method comprising:
detecting electrical signals of a patient by two or more electrodes connected by a single connecting wire and placed on the patient;
receiving the electrical signals by two or more transmitters connected to the two or more electrodes; and
wirelessly transmitting the electrical signals by the two or more transmitters.

2. The method of claim 1, wherein the two or more transmitters are universal transmitters capable of connecting to any of the two or more electrodes.

3. The method of claim 1, wherein wirelessly transmitting the electrical signals by the two or more transmitters comprises transmitting the electrical signals to an electrocardiogram monitoring device.

4. The method of claim 1, further comprising determining, by the two or more transmitters, the identity of the two or more electrodes.

5. The method of claim 4, wherein determining the identity of the two or more electrodes comprises observing a voltage on a 4 bit coded identity of the electrodes.

6. The method of claim 4, further comprising transmitting the identity of the two or more electrodes.

7. A system comprising:
two or more electrodes connected by a single connecting wire and placed on a patient, wherein the two or more electrodes are operable to detect electrical signals of the patient; and
two or more transmitters connected to the two or more electrodes, wherein the two or more electrodes are operable to:
receive the electrical signals, and
wirelessly transmit the electrical signals.

8. The system of claim 7, wherein the two or more transmitters are universal transmitters capable of connecting to any of the two or more electrodes.

9. The system of claim 7, wherein to wirelessly transmit the electrical signals comprises to transmit the electrical signals to an electrocardiogram monitoring device.

10. The system of claim 7, wherein the two or more electrodes are further operative to determine the identity of the two or more electrodes.

11. The system of claim 10, wherein to determine the identity of the two or more electrodes comprises to observe a voltage on a 4 bit coded identity of the electrodes.

12. The system of claim 10, wherein the two or more electrodes are further operative to transmit the identity of the two or more electrodes.

13. The system of claim 7, the two or more electrodes comprising:
a substrate; and
a conductive ink printed on the substrate, the conductive ink comprising:
a sensor conductor to detect the electrical signals of the patient the electrode is connected to,
a sensor conductor lead, and
a connecting wire lead.

14. The system of claim 7, further comprising a reference electrode operable to provide a reference voltage to the two or more electrodes.

15. A system comprising:
a memory storage; and
a processing unit coupled to the memory storage, wherein the processing unit is operative to:
detect electrical signals of a patient by two or more electrodes connected by a single connecting wire and placed on the patient;
receive the electrical signals by two or more transmitters connected to the two or more electrodes; and
wirelessly transmit the electrical signals by the two or more transmitters.

16. The system of claim 15, wherein the two or more transmitters are universal transmitters capable of connecting to any of the two or more electrodes.

17. The system of claim 15, wherein to wirelessly transmit the electrical signals by the two or more transmitters comprises to transmit the electrical signals to an electrocardiogram monitoring device.

18. The system of claim 15, wherein the processing unit is further operative to determine the identity of the two or more electrodes.

19. The system of claim 18, wherein to determine the identity of the two or more electrodes comprises to observe a voltage on a 4 bit coded identity of the electrodes.

20. The system of claim 18, wherein the processing unit is further operative to transmit the identity of the two or more electrodes.
